(19) United States
(12) Patent Application Publication
(10) Pub. No.: US 2026/0263799 A1
(43) Pub. Date: Sep. 10, 2026

(54) System and Method for Inserting an Electrode Lead into a Cochlea

(71) Applicants: Martin GROSSÖHMICHEN, Nienhagen (DE); Manuel KOHL, Hannover (DE); Tim NAUWELAERS, Hannover (DE); Advanced Bionics, LLC, Valencia, CA (US)

(72) Inventors: Martin Grossoehmichen, Nienhagen (DE); Manuel Christoph Kohl, Hannover (DE); Tim Nauwelaers, Hannover (DE)

(21) Appl. No.: 18/876,436

(22) PCT Filed: Jun. 27, 2022
(86) PCT No.: PCT/EP2022/067549

§ 371 Date: Dec. 18, 2024

(51) Int. Cl. A61N 1/36; A61N 1/05

(52) U.S. Cl. CPC A61N 1/36039; A61N 1/0541

(57) ABSTRACT

There is provided a system for inserting an electrode lead into a cochlea of a patient. The system comprises a vibration generator unit configured to be temporarily attached to the head of the patient so as to transduce vibrations into the cochlea during an insertion procedure of the electrode lead into the cochlea for reducing friction of the electrode lead in the cochlea and a control unit for controlling the vibration generator unit according to input from a user interface and/or input from a sensor unit for sensing the insertion of the electrode lead into the cochlea of the patient.

The disclosure relates to a system and method for inserting an electrode lead into a cochlea of a patient.

The preservation of residual hearing has become an important goal of surgery of cochlea implants (CI) since the indication range of cochlea implants has been extended more recently to patients with considerable residual hearing at some frequencies before surgery. Therefore, gentle CI electrode lead insertions are desired to minimize intra-cochlea trauma which could result in a loss of residual hearing.

Different concepts of electrode arrays with trauma-reduced insertion characteristics have been established or investigated in the past, including reducing dimensions, implementing design aspects or elements to increase the flexibility of the electrode array (for example, zig-zag wires or modified cross-section geometry), implementing design aspects to avoid movements towards the fragile basilar membrane (for example, asymmetric cross-section geometry), implementing advanced mechanical properties (for example, providing sections with different stiffness) or providing coatings to reduce friction forces.

However, none of these approaches has provided for a completely satisfactory solution, so that there is still a risk of intra-cochlea trauma and loss of residual hearing after CI surgery in mechanical practice. Further, all of these approaches have one common strong limitation in that the trauma-reducing feature of the electrode lead is always present, in particular during the entire insertion process and in all patients. This may be disadvantageous for several reasons: A too flexible electrode lead can limit the achievable insertion depth depending on the patient-specific cochlea geometry, and a permanently reduced friction could in some cases increase the backing-out effect, namely, an unwanted shift of the electrode array outside the cochlea after final placement, depending on the specific cochlea anatomy.

A different approach is known from U.S. Pat. No. 10,118,028 B2 and U.S. Pat. No. 10,022,534 B2, respectively, wherein the insertion tool, such as a forceps, used for holding the electrode lead during the insertion procedure is provided with a vibration generator, so that the electrode leads can be vibrated during the insertion procedure. The resulting vibration of the cochlea lead results in reduction of the sliding frictional force of the electrode lead within the cochlea. According to U.S. Pat. No. 10,022,534 B2 the insertion tool may be provided with a sensor positioned near the distal end of the housing of the tool for sensing a force applied to the handle of the tool, and the vibration generator may be configured to control vibration parameters based on the sensed force. According to U.S. Pat. No. 10,118,028 B2 the insertion tool may comprise a user interface which allows the surgeon to select a vibration profile from a plurality of predefined vibration profiles or to adjust the vibration profile manually based on the specific circumstances of the surgery.

It is an objective of the disclosure to provide for a system for inserting an electrode lead into a cochlea of a patient wherein the risk of intra-cochlea trauma is reduced. It is a further objective to provide for a corresponding insertion method.

According to some embodiments of the disclosure, these objectives are achieved by a system as defined in claim 1 and a method as defined in claim 27, respectively.

The present disclosure is beneficial in that, by providing a vibration generator unit which can be temporarily attached to the head of the patient during the insertion procedure vibrations can be transduced into the cochlea during the insertion procedure in a predefined and reliable manner, so as to optimize the friction reduction effect of the vibrations. This is in particular also an improvement with regard to the vibrating insertion tool described in U.S. Pat. No. 10,118,028 B2 and U.S. Pat. No. 10,022,534 B2, wherein the vibration may be accidentally dampened or altered by hand movements of the surgeon during the insertion procedure, what would result in a less effective friction reduction.

In some embodiments, the control unit may control the vibration generator unit according to input from a sensor unit for sensing the insertion of the electrode lead into the cochlea. Thereby the vibration characteristics may be automatically optimized depending on the present insertion conditions and parameters.

Some embodiments are defined in the dependent claims.

Examples of the disclosure are illustrated by reference to the drawings, wherein:

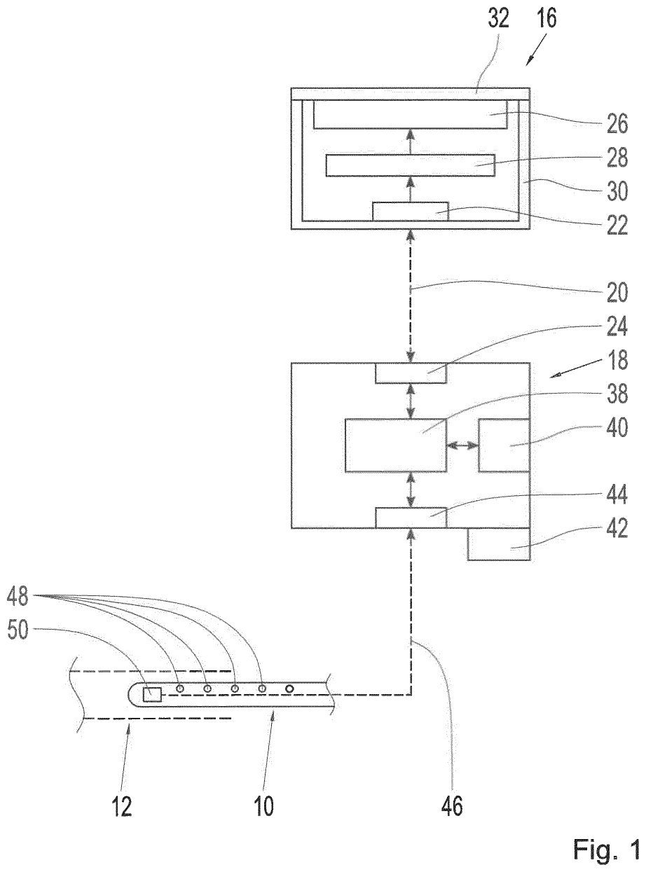

FIG. 1 is a schematic representation of an example of a system for inserting an electrode lead into a cochlea;

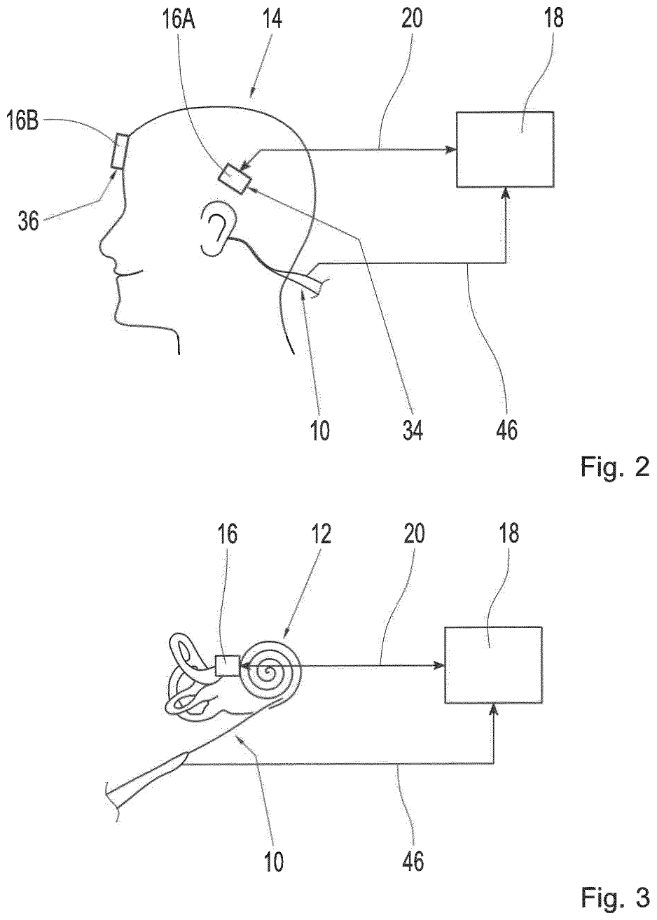

FIG. 2 illustrates an example wherein a vibration generator unit is attached to the skull of a patient; and

FIG. 3 illustrates an example wherein a vibration generator unit is attached to the promontory of a patient.

The drawings have not necessarily been drawn to scale. Similarly, some components and/or operations may be separated into different blocks or combined into a single block for the purposes of discussion of some of the embodiments of the disclosure. Moreover, while the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

FIG. 1 schematically illustrates an example of a system for inserting an electrode lead 10 into a cochlea 12 of a head 14 of a patient. The system comprises a vibration generator unit 16 and a control unit 18, which are communicatively coupled via a communication link 20, which may be wired or wireless and which is established between a communication interface 22 of the vibration generator unit 16 and a communication interface 24 of the control unit 18.

The vibration generator unit 16 comprises at least one vibration transducer 26 and a transducer driver 28 for driving the transducer 26 according to signals received via the communication interface 22 from the control unit 18. The vibration transducer 26 may be implemented as an electromotor, in particular an electromotor linked to an unbalanced mass, a hydraulic actuator, a pneumatic actuator, a mechanical actuator, an electromagnetic actuator, a piezo actuator or a sonic transducer or a combination thereof. The vibration generator unit 16 further comprises a housing 30 containing the transducer 26, the driver 28 and the communication interface 22. The housing 30 may comprises an attachment region 32 for temporarily attaching the housing 30 to the head 14 of a patient, so as to transduce vibrations of the transducer 26 into the cochlea 12 during an insertion procedure of the electrode lead 10 into the cochlea 12 for reducing friction of the electrode lead 10 within the cochlea 12.

According to one example, the vibration generator unit 16 may be configured to be temporarily attached to the skull of the patient, in particular to the temporal bone or the forehead (see FIG. 2). The vibration generator unit 16 may be attached to the skull by screwing, clamping or gluing.

According to another example, the vibration generator unit 16 may be configured to be temporarily attached to the promontory of the patient (see FIG. 3). In particular, the vibration generator unit 16 may be temporarily attached to the promontory by pressing, clamping or gluing.

The system may comprise a single vibration generator unit 16, as illustrated in FIG. 1, or it may comprise a plurality of vibration generator units, in which case each generator unit may be configured to be temporarily attached to a different location of the head 14 of the patient, such as in a first position 34 at the temporal bone and a second position 36 at the forehead, with a vibration generator unit 16A being shown in the first position 34 and a second vibration generator unit 16B being shown at the second location 36 in FIG. 2. A third vibration generator unit 16 then also may be attached to the promontory as shown in FIG. 3.

The control unit 18 comprises a processing unit 38 and a memory 40 (or is connected to an external database) and a user interface 42 allowing the surgeon to interact with the control unit 18. The control unit 18 further comprises an interface 44 for establishing a communication link 46 with the electrode lead 10 which comprises a plurality of electrodes 48 and a sensor 50 for sensing conditions during the insertion of the electrode lead 10 into the cochlea 12.

In one example, the sensor unit 50 is integrated within the electrode lead 10, such as at a distal location, as indicated in FIG. 1. The sensor unit 50 may include an optical sensor, which may act as an endoscope, so as to detect that the electrode lead 10 approaches a certain situation/condition, such as a narrow passage of the cochlea 12. Alternatively or in addition, the sensor unit 50 may comprise an acoustic or ultrasound sensor which may detect that the cochlea lead approaches a certain situation/condition, such as a narrow passage of the cochlea 12. Alternatively or in addition, the sensor unit 50 may comprise a force sensor which may detect that a certain force threshold is reached at a specific region of the electrode lead. Alternatively or in addition, the sensor unit 50 may comprise at least one strain sensor, which may be a strain gauge or a fiber Bragg grating sensor. Alternatively or in addition, the sensor unit 50 may comprise one or more of the electrodes 48, so as to detect cochlea microphonic (CM, ECohG) signals and/or electrical impedances of the respective electrodes when inserted within the cochlea 12. For example, a drop in the CM signals could indicate that the electrode lead 10 is stuck within the cochlea 12. Alternatively or in addition, the sensor unit 50 may act as an insertion depth sensor, which implementation is particularly useful in combination with a predicted virtual cochlea model obtained from, for example, pre-operative images, in particular CT images, of the cochlea, which model is implemented in the control unit 18.

The signals from the sensor unit 50 are communicated via the communication link to the control unit 18 for being taken into account by the processing unit 38 when controlling the vibration generator unit(s) 16.

In general, the control unit 18 may control the vibration generator unit(s) 16 according to input from the user interface 42 and/or input from the sensor unit(s) 50 provided via the communication link 46. The control of the vibration generator unit(s) 16 may be achieved in many different ways.

For example, the control unit 18 may control the vibration generator unit 16 based on predefined rules, taking into account the input from the sensor unit(s) 50. For example, the vibration of the vibration generator unit 16 may be activated and modified according to a threshold-based decision tree.

In some implementations, the control unit 18 may control the vibration generator unit 16 based on predefined vibration profiles, each of which consists of a set of vibration parameters. The set of vibration parameters may include, for example, at least one of vibration amplitude, vibration frequency spectrum, vibration axes orientation, vibration duration, and variation of amplitude, frequency spectrum and/or vibration axes orientation as a function of time. The control unit 18 may automatically select, taking into account the input from the sensor unit 50, one of the predefined vibration profiles or blend, taking into account the input from the sensor unit 50, two or more of the predefined vibration profiles. The predefined vibration profiles may be stored in the memory/database 40. In particular, the control unit 18 may automatically start or stop application of the respective vibration profile(s), taking into account the input from the sensor unit 50.

In some implementations, the control unit 18 may apply a machine learning procedure for periodical classification of the present situation/conditions of the insertion of the electrode lead 10 based on the input from the sensor unit 50.

In some implementations the control unit 18 may continuously adapt vibration parameters according to a machine learning procedure based on the input from the sensor unit 50. For example, the machine learning procedure may be implemented as a pre-trained artificial neural network.

In some implementations, the surgeon may manually activate/stop and modify the vibration applied by the vibration generator unit 16 via the user interface 42 of the control unit 18.

The information provided by the sensor unit(s) 50 is representative of the situation/conditions presently encountered by the electrode lead 10, so that the control unit 18 may immediately react to changes in the situation/conditions encountered by the electrode lead 10, as detected from the sensor signals received via the communication link 46, by directing the vibration generator unit 16 to adapt the vibrations applied to the skull/promontory in a suitable way.

For example, a drop in the cochlea microphonic signals may indicate that the electrode lead 10 is stuck in the cochlea 12, so that the vibration produced by the vibration generator unit 16 may be adjusted accordingly to promote release of the electrode lead.

According to a further example, the control unit 18 may direct the vibration generator unit 16 to adjust the vibration in a manner so as to achieve maximum friction reduction once the sensor unit 50 detects that a narrow passage of the cochlea is achieved (by detecting signals of an optical sensor and/or an acoustic sensor) or once a certain threshold force is detected.

According to one example, the control unit 18 may use a virtual model of the patient's cochlea 12 predicted from, for example, pre-operative CT images, wherein the information on the individual cochlea anatomy (e.g. height profile versus insertion depth) may allow to optimize the vibration characteristics depending on the insertion depth (which may be sensed by the sensor unit 50) by means of model-derived parameters (for example, distance to the lateral wall of the cochlea).

In some implementations, the vibration generator unit 16 may be configured to generate vibration at frequencies within a range of up to 100 kHz with amplitudes within a range of up to 1 mm and with different vibration axes, in particular vibrations in at least two orthogonal directions.

In some implementations, the control unit 18 may control a duration, an amplitude, an envelope and/or a repetition rate of the vibrations generated by the vibration generator unit 16. The control unit 18 may control the spectral composition of the vibrations, in particular a center frequency, a bandwidth, an amplitude as a function of frequency and a frequency modulation. Further, the control unit 18 may control the direction of the vibrations, such as longitudinal or transversal, and it may control an application site of the vibrations. The latter applies in particular when the control unit 18 controls a plurality of spaced apart vibration generator units 16 (forehead, temporal bone and/or promontory).

The control unit 18 also may take into account relevant clinical background information regarding the patient, for example, whether or not the patient has been previously implanted or whether or not ossification is present, when controlling the vibration generator unit(s) 16.

The system may be used with any type of electrode leads, and it is suitable both for traditional free-hand electrode insertion procedures or for procedures combined with a motorized and/or automated electrode insertion. The information provided by the sensor unit(s) 50 also may be used by such automated electrode insertion control.

It is also noted that the system may be used with any type of insertion tool for holding the electrode lead, including vibrating insertion tools as described in U.S. Pat. No. 10,118,028 B2 or U.S. Pat. No. 10,022,534 B2.

The phrases “in some implementations,” “according to some implementations,” “in the implementations shown,” “in other implementations,” and generally mean the particular feature, structure, or characteristic following the phrase is included in at least one implementation of the disclosure, and may be included in more than one implementation. In addition, such phrases do not necessarily refer to the same embodiments or different implementations.

The above detailed description of examples of the disclosure is not intended to be exhaustive or to limit the disclosure to the precise form disclosed above. While specific examples for the disclosure are described above for illustrative purposes, various equivalent modifications are possible within the scope of the disclosure, as those skilled in the relevant art will recognize. For example, while processes or blocks are presented in a given order, alternative implementations may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed or implemented in parallel, or may be performed at different times. Further any specific numbers noted herein are only examples: alternative implementations may employ differing values or ranges.

1. A system for inserting an electrode lead into a cochlea of a patient, comprising:
a vibration generator unit configured to be temporarily attached to the head of the patient so as to transduce vibrations into the cochlea during an insertion procedure of the electrode lead into the cochlea for reducing friction of the electrode lead in the cochlea; and
a control unit for controlling the vibration generator unit according to input from a user interface and/or input from a sensor unit for sensing the insertion of the electrode lead into the cochlea of the patient.

2. The system of claim 1, wherein the vibration generator unit is configured to be temporarily attached to the skull of the patient, in particular to the temporal bone or the forehead.

3. The system of claim 2, wherein the vibration generator unit is configured to be temporarily attached to the skull of the patient by screwing, clamping or gluing.

4. The system of claim 1, wherein the vibration generator unit is configured to be temporarily attached to the promontory of the patient.

5. The system of claim 4, wherein the vibration generator unit configured to be temporarily attached to the promontory of the patient by pressing, clamping or gluing.

6. The system of claim 1, wherein the system comprises a plurality of the vibration generator units.

7. The system of claim 6, wherein each generator unit is configured to be temporarily attached at a different location of the head of the patient.

8. The system of claim 1, wherein the vibration generator unit comprises at least one vibration transducer.

8. The system of claim 8, wherein the vibration transducer comprises at least one of an electromotor, such as an electromotor linked to an unbalanced mass, a hydraulic actuator, a pneumatic actuator, a mechanical actuator, an electromagnetic actuator, a piezo actuator, and a sonic transducer.

9. The system of claim 1, wherein the control unit is external to the vibration generator unit and wherein control und and the vibration generator unit are coupled via a wired or wireless communication interface.

11. The system of claim 1, wherein the control unit is configured to control the vibration generator unit based on predefined rules, taking into account the input from the sensor unit.

12. The system of claim 1, wherein the control unit is configured to control the vibration generator unit based on predefined vibration profiles, each of which consists of a set of vibration parameters.

13. The system of claim 12, wherein the set of vibration parameters includes at least one of: amplitude, frequency spectrum, vibration orientation, duration, and variation of amplitude, frequency spectrum and/or vibration orientation as a function of time.

14. The system of claim 12, wherein the control unit is configured to automatically select, taking into account the input from the sensor unit, one pre-defined vibration profile from the plurality of predefined vibration profiles or blend, taking into account the input from the sensor unit, pre-defined vibration profiles from a plurality of predefined vibration profiles.

15. The system of claim 1, wherein the control unit is configured to apply a machine learning procedure for periodical classification of the present electrode lead insertion situation based on the input from the sensor unit.

16. The system of claim 1, wherein the control unit is configured to continuously adapt vibration parameters according to a machine learning procedure based on the input from the sensor unit.

17. The system of claim 15, wherein the machine learning procedure is implemented as a pre-trained artificial neural network.

18. The system of claim 1, wherein the control unit is configured to control a duration, an amplitude, an envelope and/or a repetition rate of the vibrations generated by the vibration generator unit.

19. The system of claim 1, wherein the control unit is configured to control the spectral composition of the vibrations generated by the vibration generator unit, in particular a center frequency, a bandwidth, an amplitude as a function of frequency, and a frequency modulation.

20. The system of claim 1, wherein the control unit configured to control a direction of the vibrations generated by the vibration generator unit.

21. The system of claim 1, wherein the control unit is configured to control an application site of the vibrations generated by the vibration generator unit.

22. The system of claim 1, wherein the vibration generator unit is configured to generate vibrations at frequencies within a range of up to 100 kHz.

23. The system of claim 1, wherein the vibration generator unit is configured to generate vibrations with amplitudes within a range of up to 1 mm.

24. The system of claim 1, wherein the vibration generator unit is configured to generate vibrations in at least two orthogonal directions.

25. The system of claim 1, wherein the input from the sensor unit includes at least one of cochlea microphonic signals, impedances measured by the electrodes of the electrode lead, signals of an optical sensor integrated in the electrode lead, signals of an acoustic sensor integrated in the electrode lead, signals of a force sensor integrated in the electrode lead, signals of a strain sensor integrated in the electrode lead and signals from an insertion depth sensor.

26. The system of claim 1, wherein the control unit is configured to use a predicted virtual cochlea model for controlling the vibrator generator unit obtained from pre-operative images of the cochlea.

27. A method for inserting an electrode lead into a cochlea of a patient, comprising:
temporarily attaching a vibration generator unit to the head of the patient;
transducing vibrations from the vibration generator unit into the cochlea during an insertion procedure of the electrode lead into the cochlea for reducing friction of the electrode lead in the cochlea; and
controlling, via a control unit, the vibration generator unit according to input from a user interface and/or input from a sensor unit for sensing the insertion of the electrode lead into the cochlea of the patient.
